(19) United States
(12) Patent Application Publication
(10) Pub. No.: US 2026/0176217 A1
(43) Pub. Date: Jun. 25, 2026

(54) INTEGRATED FISCHER-TROPSCH AND LINEAR ALKYLBENZENE COMPLEX

(71) Applicant: UOP LLC, Des Plaines, IL (US)

(72) Inventors: Jan De Ren, Arlington Heights, IL (US); Raghavan Devanathan, New Delhi (IN); Mohamed Shakur, Hoffman Estates, IL (US); Sakthivelan Maadasamy Durai, Gurgaon (IN)

(21) Appl. No.: 19/416,413

(22) Filed: Dec. 11, 2025

(30) Foreign Application Priority Data
Dec. 20, 2024 (IN) 202411101453

(51) Int. Cl. C07C 2/66

(52) U.S. Cl. CPC C07C 2/66

(57) ABSTRACT

Processes for converting Fischer-Tropsch products into linear alkyl benzene (LAB) are described. By selecting a portion of the Fischer-Tropsch product stream, it can be fed to the alkylation reaction zone. Some pretreatment steps can be eliminated or reduced. Unreacted paraffins can be dehydrogenated and recycled to the alkylation reaction zone.

### CROSS-REFERENCES TO RELATED APPLICATIONS

This application claims priority to Indian Provisional Patent Applicant No. 202411101453 filed on Dec. 20, 2024, the entire disclosure of which is incorporated herein by way of reference.

### BACKGROUND

Existing processes for producing linear alkylated benzene (LAB) involve a number of process units. For example, in one process, a feed stream comprising kerosene is pre-fractionated to obtain a cut consistent with the carbon range of the final LAB product, i.e., C₁₀ to C₁₃ hydrocarbons. The cut is hydrotreated to remove sulfur and nitrogen and to saturate the aromatics and olefins in the feed. The normal paraffins in the hydrotreated feed are separated, and the rest of the stream is recycled to the original feed source. The normal paraffins are dehydrogenated to mono-olefins having the corresponding carbon number and selectively hydrogenates diolefins. Aromatics are adsorbed from the dehydrogenated stream. Benzene is alkylated with the mono-olefins; some transalkylation also occurs.

The Fischer-Tropsch process converts synthesis gas (carbon monoxide and hydrogen) into hydrocarbons. Some Fischer-Tropsch processes can utilize carbon dioxide and hydrogen; this process produces a higher level of olefins (e.g., about 2% olefins v. about 10 to 30% olefins) than a standard Fischer-Tropsch process.

Fischer-Tropsch synthesis is known to yield a broad mixture of products including primarily paraffins, and some olefins. The individual compounds of such mixture can contain up to about 200 carbons. Typically, the number of carbons is between about 5 and about 100, with an average number of carbons of about 60. Some Fischer-Tropsch processes yield mixtures enriched with C₅-C₃₀ alkanes containing a significant quantity of olefins and oxygenated compounds, such as alcohols. Trace amounts of sulfur-containing or nitrogen-containing products or aromatic compounds can be also present. Fischer-Tropsch products can be divided into light oils with carbon number 5-16, heavy oils with carbon numbers 7-26, and waxes with carbon numbers 12-100. Fischer-Tropsch liquids are frequently used as a raw material for obtaining various fuel and chemical products, such as, e.g., distillates such as kerosene or diesel fuels, solvents and waxes for food processing among others.

There is a need for improved processes for producing linear alkylated benzene.

### BRIEF DESCRIPTION OF THE DRAWINGS

FIG. 1 is an illustration of one embodiment of a process according to the present invention.

FIG. 2 is an illustration of another embodiment of a process according to the present invention.

### DESCRIPTION

The present invention meets this need by providing processes for converting Fischer-Tropsch products into LAB. By selecting a portion of the Fischer-Tropsch product stream, several process units from a typical LAB process can be eliminated. A Fischer-Tropsch product from a Fischer-Tropsch reaction zone does not require hydrotreating; only a deoxygenation unit is needed. In some embodiments, the Fischer-Tropsch process produces a high purity paraffin rich product so that the unit for separating the normal paraffins from the rest of the stream can be eliminated. In some case, with a high purity paraffin feed, the Fischer-Tropsch feed can be sent to dehydrogenation unit. In other embodiments, the stream from the deoxygenation unit can be fed to the dehydrogenation unit. High purity means the C₁₀ to C₁₃ paraffinic heart cut is greater than or equal to 98.5 wt % purity (approximately the same number in vol % as well) in order to meet the greater than or equal to 92% linearity specification for the final LAB product.

In some embodiments, the normal paraffin separation unit can be smaller in size. However, if the normal paraffin separation unit is not present, the n-C₁₀ to n-C₁₃ purity will only be about 88% to 90% upon fractionation. The remaining portion of the stream, which contains isoparaffins, cyclo paraffins and aromatics may be blended to form sustainable aviation fuel.

For oxygenate removal, any suitable deoxygenation only process can be used. Dehydration technologies may be used for oxygen removal because the majority of the oxygenates are alcohols. Liquid-liquid extraction with methanol/water could also be used. Hydrogenation technologies may be used by adding hydrogen to remove oxygenates in the form of water, but with some degree of olefin saturation.

In some embodiments, with a linear olefin rich product from the Fischer-Tropsch process, the feed can be sent directly to the alkylation unit. The olefins do not need to be of very high purity (as low as about 5 wt %) with some n-paraffins allowed. The alkylation will be exothermic, and some isomerization reactions will also take place. Therefore, some paraffins (greater than or equal to 30 wt %) and benzene (greater than or equal to 40 wt %) are needed to dilute the feed and limit the side reactions.

Only the unreacted paraffins from the alkylation reaction zone are dehydrogenated and recycled to the alkylation reaction zone.

One aspect of the invention is a process for producing linear alkylated benzene. In one embodiment, the process comprises providing a Fischer-Tropsch product stream comprising C₁₀-C₁₃ normal paraffins. The Fischer-Tropsch product stream is alkylated with benzene in an alkylation reaction zone comprising an alkylation reactor in the presence of an alkylation catalyst to produce an alkylated effluent stream. The alkylated effluent stream is separated into an LAB product stream comprising linear alkyl benzene and a recycle stream comprising unreacted paraffins.

In some embodiments, the Fischer-Tropsch product stream comprises greater than or equal to 30 wt % of C₁₀-C₁₃ normal paraffins, or greater than or equal to 40 wt %, or greater than or equal to 50 wt %, or greater than or equal to 60 wt %, or greater than or equal to 70 wt %, or greater than or equal to 80 wt %, or greater than or equal to 90 wt %, or greater than or equal to 95 wt %, or greater than or equal to 96 wt %, or greater than or equal to 97 wt %, or greater than or equal to 98 wt %, or greater than or equal to 98.5 wt %.

In some embodiments, the Fischer-Tropsch product stream comprises less than or equal to 30 wt % of C₁₀-C₁₃ iso-paraffins, or less than or equal to 25 wt %, or less than or equal to 20 wt %, or less than or equal to 15 wt %, or less than or equal to 10 wt %, or less than or equal to 5 wt %, or less than or equal to 4 wt %, or less than or equal to 3 wt %, or less than or equal to 2 wt %, or less than or equal to 1.5 wt %.

In some embodiments, providing the Fischer-Tropsch product stream comprises: separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins. The non-normal paraffins can be returned to the refinery or used for blending in the diesel pool.

In some embodiments, the process further comprises: dehydrogenating the recycle stream in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins; and passing the dehydrogenated effluent stream to the alkylation reaction zone.

In some embodiments, the process further comprises introducing a paraffin stream comprising 98.5 wt % or more of C₁₀-C₁₃ normal paraffins into the dehydrogenation reaction zone.

In some embodiments, the paraffin stream comprises 85 wt % or less of a total amount of the recycle stream and the paraffin stream in the dehydrogenation reaction zone.

In some embodiments, the process further comprises prefractionating the Fischer-Tropsch product stream; or deoxygenating the Fischer-Tropsch product stream; or dehydrating the Fischer-Tropsch product stream; or hydrogenating the Fischer-Tropsch product stream; or combinations thereof to produce a treated Fischer-Tropsch product stream; and wherein alkylating the Fischer-Tropsch product stream comprises alkylating the treated Fischer-Tropsch product stream.

In some embodiments, the process further comprises feeding the treated Fischer-Tropsch product stream to a dehydrogenation reaction zone before alkylating the treated Fischer-Tropsch product stream.

In some embodiments, the LAB product stream has a linearity of greater than or equal to 70%, or greater than or equal to 80%, or greater than or equal to 85%, or greater than or equal to 90%, or greater than or equal to 91%, or greater than or equal to 92%, or greater than or equal to 93%, or greater than or equal to 95%, or greater than or equal to 95%.

In some embodiments, separating the alkylated effluent stream into the LAB product stream and the recycle stream comprises separating the alkylated effluent stream into the LAB product stream, the recycle stream, and a byproduct stream comprising heavy alkyl benzene, further comprising: passing at least a portion of the byproduct stream to a reforming reaction zone or a gasification reaction zone of Fischer Tropsch based fuel production complex; or passing at least a portion of the byproduct stream to a transalkylation reaction zone; or both.

Another aspect of the invention is a process for producing linear alkylated benzene. In one embodiment, the process comprises providing a Fischer-Tropsch product stream comprising C₁₀-C₁₃ normal paraffins. The Fischer-Tropsch product stream is alkylated with benzene in an alkylation reaction zone comprising an alkylation reactor in the presence of an alkylation catalyst to produce an alkylated effluent stream. The alkylated effluent stream is separated into an LAB product stream comprising linear alkyl benzene and a recycle stream comprising unreacted paraffins, wherein the LAB product stream has a linearity of at least 70%. The recycle stream is dehydrogenated in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins. A paraffin stream comprising greater than or equal to 98.5 wt % of C₁₀-C₁₃ normal paraffins is introduced into the dehydrogenation reaction zone. The dehydrogenated effluent stream is passed to the alkylation reaction zone.

In some embodiments, the Fischer-Tropsch product stream comprises 30 wt % or more of C₁₀-C₁₃ normal paraffins. In some embodiments, the Fischer-Tropsch product stream comprises less than or equal to 30 wt % of C₁₀-C₁₃ iso-paraffins.

In some embodiments, providing the Fischer-Tropsch product stream comprises: separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins.

In some embodiments, the paraffin stream comprises 85 wt % or less of a total amount of the recycle stream and the paraffin stream in the dehydrogenation reaction zone.

In some embodiments, the process further comprises: prefractionating the Fischer-Tropsch product stream; or deoxygenating the Fischer-Tropsch product stream; or dehydrating the Fischer-Tropsch product stream; or hydrogenating the Fischer-Tropsch product stream; or combinations thereof to produce a treated Fischer-Tropsch product stream; and wherein alkylating the Fischer-Tropsch product stream comprises alkylating the treated Fischer-Tropsch product stream.

In some embodiments the process further comprises: feeding the treated Fischer-Tropsch product stream to a dehydrogenation reaction zone before alkylating the treated Fischer-Tropsch product stream.

In some embodiments, separating the alkylated effluent stream into the LAB product stream and the recycle stream comprises separating the alkylated effluent stream into the LAB product stream, the recycle stream, and a byproduct stream comprising heavy alkyl benzene, further comprising: passing at least a portion of the byproduct stream to a reforming reaction zone or gasification reaction zone of a Fischer Tropsch based fuel production complex; or passing at least a portion of the byproduct stream to a transalkylation reaction zone; or both.

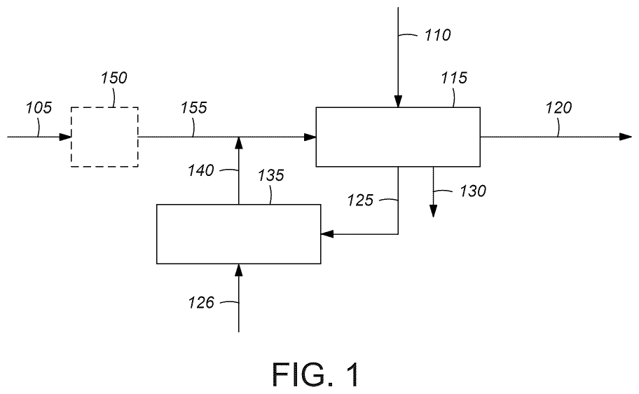

FIG. 1 illustrates one embodiment of the process 100 for producing linear alkylated benzene. The Fischer-Tropsch product stream 105 from a Fischer-Tropsch reaction zone (not shown) and a benzene stream 110 are sent to the alkylation reaction zone 115 comprising an alkylation reactor in the presence of an alkylation catalyst to form a reaction mixture comprising LAB, unreacted paraffins, and heavy alkylbenzene byproducts.

The Fischer-Tropsch product stream may comprise C₁₀-C₁₃ normal paraffins and some olefins, along with small amounts of isoparaffins and cycloparaffins in some cases.

Any suitable alkylation catalyst for producing LAB can be used. Suitable alkylation catalysts include, but are not limited to, aluminum chloride, hydrogen fluoride, silica alumina, or zeolitic catalysts.

Any suitable reaction conditions for producing LAB can be used. These alkylation conditions include a temperature in the range between about 80° C. and 140° C., most usually at a temperature not exceeding 135° C. Since the alkylation is conducted as a liquid phase process, pressures must be sufficient to maintain the reactants in the liquid state. The requisite pressure necessarily depends upon the feedstock and temperature, but normally is in the range of 200-1000 psig (1379-6895 kPa), and most usually 300-500 psig (2069-3448 kPa).

The alkylation reaction mixture is separated into an LAB stream 120, an unreacted paraffin stream 125, and a byproduct stream 130. The LAB stream 120 is recovered.

All or a portion of the byproduct stream 130 can be sent to a Fischer-Tropsch based fuel production complex, or a transalkylation reaction zone, for example, for further processing.

The unreacted paraffin stream 125 is sent to a dehydrogenation reaction zone 135 comprising a dehydrogenation reactor to form olefins. A paraffin stream 126 is introduced into the dehydrogenation reaction zone 135.

The dehydrogenation effluent stream 140 is sent to the alkylation reaction zone 115.

In some embodiments, the Fischer-Tropsch product stream 105 can be sent to one or more optional reaction zones and/or fractionation zones 150 before being sent to the alkylation reaction zone 115. In this case, the treated Fischer-Tropsch product stream 155 is then passed to the alkylation reaction zone 115.

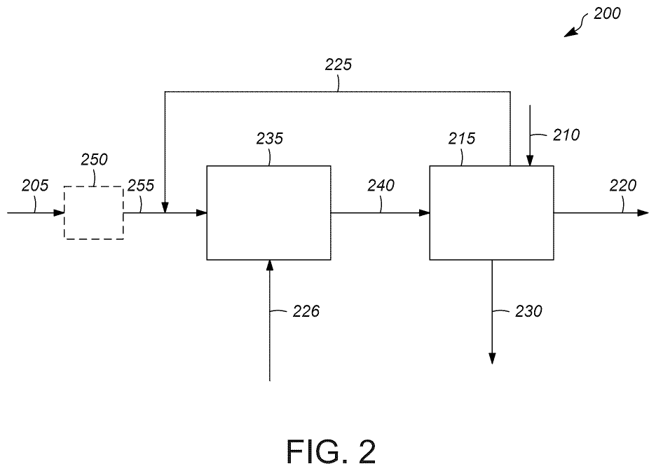

FIG. 2 illustrates another embodiment of the process 200 for producing linear alkylated benzene. The Fischer-Tropsch product stream 205 from a Fischer-Tropsch reaction zone (not shown) is sent to a dehydrogenation reaction zone 235 comprising a dehydrogenation reactor to form olefins.

The Fischer-Tropsch product stream may comprise C₁₀-C₁₃ normal paraffins and some olefins, along with small amounts of isoparaffins and cycloparaffins in some cases.

A paraffin stream 226 is introduced into the dehydrogenation reaction zone 235.

The dehydrogenation effluent stream 240 and a benzene stream 210 are sent to the alkylation reaction zone 215 comprising an alkylation reactor in the presence of an alkylation catalyst to form a reaction mixture comprising LAB, unreacted paraffins, and heavy alkylbenzene byproducts.

Any suitable alkylation catalyst for producing LAB can be used. Suitable alkylation catalysts include, but are not limited to, aluminum chloride, hydrogen fluoride, silica alumina, or zeolitic catalysts.

Any suitable reaction conditions for producing LAB can be used. These alkylation conditions include a temperature in the range between about 80° C. and 140° C., most usually at a temperature not exceeding 135° C. Since the alkylation is conducted as a liquid phase process, pressures must be sufficient to maintain the reactants in the liquid state. The requisite pressure necessarily depends upon the feedstock and temperature, but normally is in the range of 200-1000 psig (1379-6895 kPa), and most usually 300-500 psig (2069-3448 kPa).

The alkylation reaction mixture is separated into an LAB stream 220, an unreacted paraffin stream 225, and a byproduct stream 230. The LAB stream 220 is recovered.

All or a portion of the byproduct stream 230 can be sent to a Fischer-Tropsch based fuel production complex, or a transalkylation reaction zone, for example, for further processing.

The unreacted paraffin stream 225 is sent to the dehydrogenation reaction zone 235.

In some embodiments, the Fischer-Tropsch product stream 205 can be sent to one or more optional reaction zones and/or fractionation zones 250 before being sent to the dehydrogenation reaction zone 235. In this case, the treated Fischer-Tropsch product stream 255 is then passed to the dehydrogenation reaction zone 235.

### EXAMPLES

### Example 1 (Corresponding to FIG. 1)

A known Fischer-Tropsch product stream 105 comprising light hydrocarbons from a cobalt based Fischer-Tropsch process is passed through a dehydration process. As estimated by the process model, the oxygenates in the product stream are removed in the form of water without losing the olefinicity of the stream to produce a treated Fischer-Tropsch product stream 155. The treated Fischer-Tropsch product stream 155 and a benzene stream are sent to the alkylation reaction zone wherein the olefins are converted to predominantly alkylated benzene as estimated by the process model, forming a reaction product comprising LAB stream 120, an unreacted paraffin stream 125, and a byproduct stream. The unreacted paraffin stream 125 and a fresh paraffin stream 126 are dehydrogenated to convert the n-paraffins to olefins as estimated by the process model, to form dehydrogenation effluent stream 140 which is sent to the alkylation reaction zone for increasing the production of LAB. The composition of the respective streams as estimated by the process model are indicated in Table 1.

TABLE 1

| | | | | | | |
|---|---|---|---|---|---|---|
| Stream # | 105 | 155 | 126 | 140 | 125 | 120 |

Component Summary (wt %)

| | | | | | | |
|---|---|---|---|---|---|---|
| n-Paraffins | 70-80% | 75-88% | >98.5% | 75-85% | >90% | |
| Aromatics | <0.1% | <0.1% | | <0.1% | <0.1% | >99.9% |
| i-Paraffins | 2-10% | 3-12% | <1.5% | 3-12% | 3-12% | |
| Olefins | 5-15% | 8-18% | | 8-18% | 0% | |
| Oxygenates | 5-15% | <0.1% | | <1% | <1% | |

### Example 2 (Corresponding to FIG. 2)

A known Fischer-Tropsch product stream 205 comprising light hydrocarbons from a cobalt based Fischer-Tropsch process is passed through a hydrogenating step involving a hydro-deoxygenation process. As estimated by the process model, the oxygenates in the Fischer-Tropsch product stream are removed in the form of water, and most of the olefins are saturated to produce treated Fischer-Tropsch product stream 255. The treated Fischer-Tropsch product stream 255 is dehydrogenated to convert the n-paraffins to olefins as estimated by the process model to form dehydrogenation effluent stream 240 which is sent to the alkylation reaction zone. With the addition of a benzene stream to the alkylation reaction zone, the olefins are converted to predominantly alkylated benzene as estimated by the process model, forming a reaction product comprising LAB stream 220, an unreacted paraffin stream 225, and a byproduct stream. The unreacted paraffin stream 225 and a fresh paraffin stream 226 are dehydrogenated to convert the n-paraffins to olefins as estimated by the process model along with the treated Fischer-Tropsch product stream 255 for increasing the production of LAB. The composition of the respective streams as estimated by the process model are indicated in Table 2.

TABLE 2

| | | | | | | |
|---|---|---|---|---|---|---|
| Stream # | 205 | 255 | 226 | 240 | 225 | 220 |

Component Summary (wt %)

| | | | | | | |
|---|---|---|---|---|---|---|
| n-Paraffins | 70-80% | >90% | >98.5% | 80-90% | >90% | |
| Aromatics | <0.1% | <0.1% | | <0.1% | <0.1% | >99.9% |
| i-Paraffins | 2-10% | 3-12% | <1.5% | 3-12% | 3-12% | |
| Olefins | 5-15% | <0.1% | | 8-18% | 0% | |
| Oxygenates | 5-15% | <0.1% | | <1% | <1% | |

### Specific Embodiments

While the following is described in conjunction with specific embodiments, it will be understood that this description is intended to illustrate and not limit the scope of the preceding description and the appended claims.

A first embodiment of the invention is a process for producing linear alkylated benzene comprising providing a Fischer-Tropsch product stream comprising C₁₀-C₁₃ normal paraffins; alkylating the Fischer-Tropsch product stream with benzene in an alkylation reaction zone comprising an alkylation reactor in the presence of an alkylation catalyst to produce an alkylated effluent stream; and separating the alkylated effluent stream into an LAB product stream comprising linear alkyl benzene and a recycle stream comprising unreacted paraffins. An embodiment of the invention is one, any or all of prior embodiments in this paragraph up through the first embodiment in this paragraph wherein the Fischer-Tropsch product stream comprises greater than or equal to 30 wt % of C₁₀-C₁₃ normal paraffins and less than or equal to 30 wt % of C₁₀-C₁₃ iso-paraffins. An embodiment of the invention is one, any or all of prior embodiments in this paragraph up through the first embodiment in this paragraph wherein providing the Fischer-Tropsch product stream comprises separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins. An embodiment of the invention is one, any or all of prior embodiments in this paragraph up through the first embodiment in this paragraph further comprising dehydrogenating the recycle stream in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins; and passing the dehydrogenated effluent stream to the alkylation reaction zone. An embodiment of the invention is one, any or all of prior embodiments in this paragraph up through the first embodiment in this paragraph further comprising introducing a paraffin stream comprising greater than or equal to 98.5 wt % of C₁₀-C₁₃ normal paraffins into the dehydrogenation reaction zone. An embodiment of the invention is one, any or all of prior embodiments in this paragraph up through the first embodiment in this paragraph wherein the paraffin stream comprises less than or equal to 85 wt % of a total amount of the recycle stream and the paraffin stream in the dehydrogenation reaction zone. An embodiment of the invention is one, any or all of prior embodiments in this paragraph up through the first embodiment in this paragraph further comprising prefractionating the Fischer-Tropsch product stream; or deoxygenating the Fischer-Tropsch product stream; or dehydrating the Fischer-Tropsch product stream; or hydrogenating the Fischer-Tropsch product stream; or combinations thereof to produce a treated Fischer-Tropsch product stream; and wherein alkylating the Fischer-Tropsch product stream comprises alkylating the treated Fischer-Tropsch product stream. An embodiment of the invention is one, any or all of prior embodiments in this paragraph up through the first embodiment in this paragraph further comprising feeding the treated Fischer-Tropsch product stream to a dehydrogenation reaction zone before alkylating the treated Fischer-Tropsch product stream. An embodiment of the invention is one, any or all of prior embodiments in this paragraph up through the first embodiment in this paragraph wherein the LAB product stream has a linearity of greater than or equal to 70. An embodiment of the invention is one, any or all of prior embodiments in this paragraph up through the first embodiment in this paragraph wherein separating the alkylated effluent stream into the LAB product stream and the recycle stream comprises separating the alkylated effluent stream into the LAB product stream, the recycle stream, and a byproduct stream comprising heavy alkyl benzene, further comprising passing at least a portion of the byproduct stream to a reforming reaction zone or a gasification reaction zone of a Fischer Tropsch based fuel production complex; or passing at least a portion of the byproduct stream to a transalkylation reaction zone; or both.

A second embodiment of the invention is a process for producing linear alkylated benzene comprising providing a Fischer-Tropsch product stream comprising C₁₀-C₁₃ normal paraffins; alkylating the Fischer-Tropsch product stream with benzene in an alkylation reaction zone comprising an alkylation reactor in the presence of an alkylation catalyst to produce an alkylated effluent stream; separating the alkylated effluent stream into an LAB product stream comprising linear alkyl benzene and a recycle stream comprising unreacted paraffins, wherein the LAB product stream has a linearity of greater than or equal to 70%; dehydrogenating the recycle stream in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins; introducing a paraffin stream comprising greater than or equal to 98.5 wt % of C₁₀-C₁₃ normal paraffins into the dehydrogenation reaction zone; and passing the dehydrogenated effluent stream to the alkylation reaction zone. An embodiment of the invention is one, any or all of prior embodiments in this paragraph up through the second embodiment in this paragraph wherein the Fischer-Tropsch product stream comprises greater than or equal to 30 wt % of C₁₀-C₁₃ normal paraffins and less than or equal to 30 wt % of C₁₀-C₁₃ iso-paraffins. An embodiment of the invention is one, any or all of prior embodiments in this paragraph up through the second embodiment in this paragraph wherein providing the Fischer-Tropsch product stream comprises separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins. An embodiment of the invention is one, any or all of prior embodiments in this paragraph up through the second embodiment in this paragraph wherein the paraffin stream comprises less than or equal to 85 wt % of a total amount of the recycle stream and the paraffin stream in the dehydrogenation reaction zone. An embodiment of the invention is one, any or all of prior embodiments in this paragraph up through the second embodiment in this paragraph further comprising prefractionating the Fischer-Tropsch product stream; or deoxygenating the Fischer-Tropsch product stream; or dehydrating the Fischer-Tropsch product stream; or hydrogenating the Fischer-Tropsch product stream; or combinations thereof to produce a treated Fischer-Tropsch product stream; and wherein alkylating the Fischer-Tropsch product stream comprises alkylating the treated Fischer-Tropsch product stream. An embodiment of the invention is one, any or all of prior embodiments in this paragraph up through the second embodiment in this paragraph further comprising feeding the treated Fischer-Tropsch product stream to a dehydrogenation reaction zone before alkylating the treated Fischer-Tropsch product stream. An embodiment of the invention is one, any or all of prior embodiments in this paragraph up through the second embodiment in this paragraph wherein separating the alkylated effluent stream into the LAB product stream and the recycle stream comprises separating the alkylated effluent stream into the LAB product stream, the recycle stream, and a byproduct stream comprising heavy alkyl benzene, further comprising passing at least a portion of the byproduct stream to a reforming reaction zone or gasification reaction zone of a Fischer Tropsch based fuel production complex; or passing at least a portion of the byproduct stream to a transalkylation reaction zone; or both.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for producing linear alkylated benzene comprising:
providing a Fischer-Tropsch product stream comprising C₁₀-C₁₃ normal paraffins;
alkylating the Fischer-Tropsch product stream with benzene in an alkylation reaction zone comprising an alkylation reactor in the presence of an alkylation catalyst to produce an alkylated effluent stream; and
separating the alkylated effluent stream into an LAB product stream comprising linear alkyl benzene and a recycle stream comprising unreacted paraffins.

2. The process of claim 1 wherein the Fischer-Tropsch product stream comprises greater than or equal to 30 wt % of C₁₀-C₁₃ normal paraffins and less than or equal to 30 wt % of C₁₀-C₁₃ iso-paraffins.

3. The process of claim 1 wherein providing the Fischer-Tropsch product stream comprises:
separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins.

4. The process of claim 1 further comprising:
dehydrogenating the recycle stream in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins; and
passing the dehydrogenated effluent stream to the alkylation reaction zone.

5. The process of claim 4 further comprising:
introducing a paraffin stream comprising greater than or equal to 98.5 wt % of C₁₀-C₁₃ normal paraffins into the dehydrogenation reaction zone.

6. The process of claim 5 wherein the paraffin stream comprises less than or equal to 85 wt % of a total amount of the recycle stream and the paraffin stream in the dehydrogenation reaction zone.

7. The process of claim 1 further comprising:
prefractionating the Fischer-Tropsch product stream; or
deoxygenating the Fischer-Tropsch product stream; or
dehydrating the Fischer-Tropsch product stream; or
hydrogenating the Fischer-Tropsch product stream; or
combinations thereof to produce a treated Fischer-Tropsch product stream; and
wherein alkylating the Fischer-Tropsch product stream comprises alkylating the treated Fischer-Tropsch product stream.

8. The process of claim 7 further comprising:
feeding the treated Fischer-Tropsch product stream to a dehydrogenation reaction zone before alkylating the treated Fischer-Tropsch product stream.

9. The process of claim 1 wherein the LAB product stream has a linearity of greater than or equal to 70.

10. The process of claim 1 wherein separating the alkylated effluent stream into the LAB product stream and the recycle stream comprises separating the alkylated effluent stream into the LAB product stream, the recycle stream, and a byproduct stream comprising heavy alkyl benzene, further comprising:
passing at least a portion of the byproduct stream to a reforming reaction zone or a gasification reaction zone of a Fischer Tropsch based fuel production complex; or
passing at least a portion of the byproduct stream to a transalkylation reaction zone;
or both.

11. A process for producing linear alkylated benzene comprising:
providing a Fischer-Tropsch product stream comprising C₁₀-C₁₃ normal paraffins;
alkylating the Fischer-Tropsch product stream with benzene in an alkylation reaction zone comprising an alkylation reactor in the presence of an alkylation catalyst to produce an alkylated effluent stream;
separating the alkylated effluent stream into an LAB product stream comprising linear alkyl benzene and a recycle stream comprising unreacted paraffins, wherein the LAB product stream has a linearity of greater than or equal to 70%;
dehydrogenating the recycle stream in a dehydrogenation reaction zone comprising a dehydrogenation reactor in the presence of a dehydrogenation catalyst to produce a dehydrogenated effluent stream comprising 5 wt % to 30 wt % C₁₀-C₁₃ mono and di olefins;
introducing a paraffin stream comprising greater than or equal to 98.5 wt % of C₁₀-C₁₃ normal paraffins into the dehydrogenation reaction zone; and
passing the dehydrogenated effluent stream to the alkylation reaction zone.

12. The process of claim 11 wherein the Fischer-Tropsch product stream comprises greater than or equal to 30 wt % of C₁₀-C₁₃ normal paraffins and less than or equal to 30 wt % of C₁₀-C₁₃ iso-paraffins.

13. The process of claim 11 wherein providing the Fischer-Tropsch product stream comprises:
separating a Fischer-Tropsch effluent stream from a Fischer-Tropsch reaction zone into the Fischer-Tropsch product stream comprising 75 wt % to 98.5 wt % C₁₀-C₁₃ normal paraffins and a raffinate stream comprising non-normal paraffins.

14. The process of claim 11 wherein the paraffin stream comprises less than or equal to 85 wt % of a total amount of the recycle stream and the paraffin stream in the dehydrogenation reaction zone.

15. The process of claim 11 further comprising:
prefractionating the Fischer-Tropsch product stream; or
deoxygenating the Fischer-Tropsch product stream; or
dehydrating the Fischer-Tropsch product stream; or
hydrogenating the Fischer-Tropsch product stream; or
combinations thereof to produce a treated Fischer-Tropsch product stream; and
wherein alkylating the Fischer-Tropsch product stream comprises alkylating the treated Fischer-Tropsch product stream.

16. The process of claim 15 further comprising:
feeding the treated Fischer-Tropsch product stream to a dehydrogenation reaction zone before alkylating the treated Fischer-Tropsch product stream.

17. The process of claim 11 wherein separating the alkylated effluent stream into the LAB product stream and the recycle stream comprises separating the alkylated effluent stream into the LAB product stream, the recycle stream, and a byproduct stream comprising heavy alkyl benzene, further comprising:
passing at least a portion of the byproduct stream to a reforming reaction zone or gasification reaction zone of a Fischer Tropsch based fuel production complex; or
passing at least a portion of the byproduct stream to a transalkylation reaction zone;
or both.
